(19) United States
(12) Patent Application Publication
(10) Pub. No.: US 2010/0198106 A1
(43) Pub. Date: Aug. 5, 2010

(54) BIOPSY FORCEPS FOR TAKING ONE OR MORE SAMPLES

(75) Inventors: John Bleibach, Hellerup (DK); Lars Schoning, Hellerup (DK)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: MULTI BIOPSY SAMPLING CO. ApS

(21) Appl. No.: 12/449,619

(22) PCT Filed: Feb. 18, 2008
(86) PCT No.: PCT/DK2008/000071

§ 371 Date: Mar. 9, 2010

(30) Foreign Application Priority Data
Feb. 19, 2007 (DK) PA 2007 00253

(51) Int. Cl. A61B 10/06

(52) U.S. Cl. 600/564

(57) ABSTRACT

The invention relates to biopsy forceps for collecting tissue samples and the like in connection with the use of some kind of examination or treatment telescope (1) in the treatment of humans or animals, wherein the part of the forceps (1) that performs cutting or shearing is provided with an exchange in the form of two mutually hinged arms (5, 9), wherein the first arm (5) comprises a holder device (6, 14) for securing a receptacle part (7, 12), which receptacle part (7, 12) is, at the end that faces away from the arm (5) in which the receptacle (7) is secured, provided with a cutting means (8), and wherein a second arm (9) which, during the sampling, cooperates with the first arm (5) with the receptacle, is provided with a kind of stop means or abutment (10) which cooperates with the cutting means (8) on the receptacle (7, 12) to cut off or shear off the tissue sample (11). The biopsy forceps (1) is provided with an exchangeable receptacle (7, 12) for storing one or more tissue samples (11).

The invention relates to biopsy forceps for taking tissue samples and the like in connection with the use of a kind of examination or treatment telescope in the treatment of humans or animals.

### PRIOR ART

In recent years it has become increasingly common to perform examinations and/or surgical interventions by means of a kind of telescope. The advantage of using an examination and/or treatment telescope is that it is necessary to make only such opening in the patient that there is room for inserting the examination and/or treatment telescope through the opening made. It is also possible to use one of the natural orifices in the body for introducing the biopsy forceps, eg by endoscopy, in particular when examining the digestive tract, optionally through the oesophagus, or when examining the digestive tract through the rectum.

In connection with the taking of samples through such examination and/or treatment telescope (in the following designated “the telescope”) biopsy forceps are known in the form of a comparatively thin and long pliable object, primarily consisting of an outer cable that extends throughout the length of the forceps, in which outer cable an internal manoeuvring cable can be conveyed that connects an operating handle in the outer end of the biopsy forceps to a set of jaws at the end of the forceps that is taken through the telescope. By influencing the operating handle the jaws can be moved in such a manner that it is possible to bite off a piece of tissue and pull the tissue out along with the biopsy forceps.

For some examinations it is necessary to collect more than one sample for subsequent analysis. To keep track of the individual samples or series of samples taken from a specific area it is an advantage if the samples taken can be kept in some kind of reservoir.

Such instrument is known from U.S. Pat. No. 4,651,753. This instrument comprises a cylindrical end with a cylindrical cutter intended for sliding back and forth within the cylindrical end of the instrument. An oval opening in the cylinder wall of the cylindrical end makes it possible to suck in tissue at the cylindrical end, following which the cylindrical cutter cuts off a tissue sample.

This means that, when a sample is to be taken, it is necessary to create a sub-atmospheric pressure that draws a portion of the tissue so far into the opening in the cylinder wall that the cylindrical cutter is able to cut off a tissue sample.

Moreover, by this instrument it is possible to take samples only from the side of the instrument and not from the end which means that it may be considerably more complex to manoeuvre the instrument in position for sample collection.

Biopsy cables are also known that have jaws positioned at the end of the cable; which jaws, however, do not perform a clean cut; rather, they seize around the tissue and rip it off.

### Novel Technique

It is the object of the invention to provide biopsy forceps capable of accommodating the samples taken in some kind of receptacle and capable of taking samples, where the samples are cut or sheared off in a clean cut without having to supply vacuum or other additional measures to introduce tissue into the sample taking equipment.

This is accomplished by providing biopsy forceps where the part of the forceps that performs the cut or shearing is provided with an exchange in the form of two mutually hinged arms, wherein the first arm comprises a holder device for securing a receptacle part, said receptacle part being, at the end that faces away from the arm in which the receptacle is secured, provided with a cutting means. Another arm that, during the sample collection, cooperates with the first arm with the receptacle is provided with some kind of stop means or abutment that combines with the cutting means on the receptacle to cut off or shear off the tissue sample.

The receptacle can be configured as a cylinder arranged and secured in a corresponding opening in the first arm. The opening for attachment of the receptacle can be closed at the bottom, but it may also be open where an edge or collar is provided along the edge of the opening at the end of the opening that faces away from the other arm. In those cases where the opening is not closed at the bottom the bottom can be replaced by some kind of sieve. The sieve may be a plate provided with a number of holes or a kind of grating. According to one embodiment of the opening, where the bottom is closed, the bottom may be provided with a number of holes.

According to another embodiment this kind of sieve may be formed at the end of the cylindrical receptacle, which end faces away from the end which is provided with cutting means.

The sieve serves the purpose of retaining the samples taken with the biopsy forceps to the effect that they do not fall out at the one end of the receptacle.

Interiorly, the receptacle may be provided with a surface that resembles sharkskin, ie provided with a row of relatively small one-way tips that protrude from the surface at an angle to the effect that samples, once they have entered the receptacle by movement along the side of the receptacle, are directed towards the end of the receptacle that faces away from the opening.

Moreover, the stop means or abutments of the biopsy forces can be caused to be above the opening of the receptacle when one has finished taking samples and hence serve as lid.

This may be accomplished by the forceps being locked in a closed position during withdrawal when no further samples are to be taken.

The abutment may be configured essentially plane, but may be provided with an elevation at the middle which, when the forceps are closed, extends into the receptacle at least past the cutting edge of the cutting means. According to a further embodiment of the abutment, it may be provided with an indentation at the middle to the effect that, when the forceps are closed, the receptacle, or at least the cutting edge of the cutting means, extends into the abutment.

When the biopsy forceps have been taken out either of the telescope or along with the telescope, the receptacle with the samples can be removed from the forceps. This may be accomplished by means of tongs, optionally adapted to that particular function. The most important aspect of such tongs for removing the receptacle is that such tongs seize securely around the receptacle to avoid that the samples are wasted or polluted eg if the receptacle is let go.

The receptacle may also be provided with engagement means that engage with cooperating means in the arm of the biopsy forceps. For instance, a bayonet socket may be used, or a thread or a tight-fitting connection optionally featuring a magnet for additional attachment. The attachment of the receptacle may also be accomplished in that the cylinder wall of the receptacle is slotted in a longitudinal direction whereby the receptacle is squeezed and hence secured in the arm on the tongs when positioned.

According to a further embodiment of the biopsy forceps, the receptacle may be formed from a kind of revolver magazine, to the effect that the magazine can be turned, following sampling, such that a new chamber is in position for a new sample to be taken and arranged in an empty chamber. This procedure can be repeated until there are no more empty chambers in the magazine.

To ensure that the samples do not fall out of the charged magazines, a disc or plate may be arranged on top of the magazine that covers the chambers in the magazine with the exception of precisely the chamber that has been positioned to receive the next sample.

According to a particular embodiment the magazine may be configured such that, at least between two chambers, there is a distance that corresponds essentially to the diameter of a chamber. Thereby the magazine may, following finished sampling, be parked in a position where precisely that area is arranged corresponding to the opening in the plate or the disc, to the effect that all of the samples that are taken remain in their respective chambers when the biopsy forceps are taken out of the telescope or are removed from the patient along with the telescope.

In connection with the manoeuvring cable, an advancement mechanism may be provided for advancing the magazine such that, when a sample has been taken, the magazine may be advanced such that the next chamber is in position for receiving a sample.

The advancement mechanism to the magazine is optionally provided with a locking mechanism that either keeps the magazine in position until the next advancement occurs or (optionally in combination) ensures that the magazine can be advanced in only one direction.

The advancement mechanism may be driven by the manoeuvring cable or some other mechanism.

According to one embodiment of the advancement mechanism, it may be configured as a relatively flat latch, leaf spring or the like that is connected to the central manoeuvring cable in the biopsy forceps. The advantages of the flat configuration are ia that the latch is thereby controlled by the tool/biopsy forceps to the effect that the latch does not capsize during activation of the advancement of the magazine.

In other cases it is an option to use a simple rigid material, eg the end of a cable can be used, optionally provided with some kind of guide or slide bushing in proximity of the point of attack, where the cable influences the magazine.

When the desired number of samples has been taken, the samples can be taken from the magazine to a storage receptacle with formalin or other tissue-preserving preparation, or the entire magazine may be dismounted and arranged in formalin or other tissue-preserving preparation, in which the tissue can be stored until further examination takes place.

It applies to all embodiments that they may be connected to a cable by means of a coupling where the forceps may be attached to the outer part of the manoeuvring cable by means of snap-coupling, bayonet socket, thread or the like, and wherein the inner cable sliding within the outer cable is secured to an intermediate piece for the arms of the forceps in the same manner as the one in which a handbrake cable is secured to the brake handle of a bike or the like.

Thereby various tools can be used in conjunction with a cable, and by a bore being configured at that end of the telescope which is inside the patient in use, which bore is larger than the passage in which the cable usually travels, it is possible to mount the tool on the cable after the cable has been taken through the passage within the telescope.

However, the invention is not restricted to that embodiment, some tools allowing, due to their size, them to travel through the passage in the telescope along with the cable after the telescope has been deployed in the patient.

By the invention according to claim 1 it is accomplished that the tissue sample taken is not torn off, but rather cut or sheared off with a clean cut.

By claim 2 it is accomplished that samples taken can be sent to examination stored in and protected by the receptacle, the latter being dismountable.

By claims 3 and 4 it is accomplished that samples taken can be sent to examination, where each sample can be associated with a specific chamber for identification or determination of when the sample was taken.

By the invention as described in claim 5 it is accomplished that the sample does not fall out of the receptacle or the chamber in which it is stored.

By claim 6 it is accomplished that the tool can be coupled to the cable after the cable has been taken through a smaller passage within the telescope, and that optional tools are available for use in connection with the same cable.

By claims 7 and 8 according to the invention, it is accomplished that liquid and the like may travel through holes or net, while the collected samples are unable to pass and hence remain in the receptacle or chamber.

By claims 9 and 10 a convenient manner is accomplished in which an empty chamber can be positioned for collecting a sample.

It is also possible to apply the technique to a capsule endoscopy where the patient is intended to swallow a capsule, which capsule passes through the intestines of the patient. During the passage, it is possible to take pictures, video and to perform scans of internal organs and the like.

By configuring such capsule with the tool described in the invention, where the tool is activated by manoeuvring means from/inside the capsule, further options are provided for performing samplings from the intestines of a patient in a convenient manner. The capsule may optionally be manoeuvred or directed through the patient by means of magnetic fields, remote control or the like wireless transfer. It is also conceivable that transfer of control signals is accomplished through a cable that is carefully pulled out of the patient again, along with the capsule, or, following ended use, the cable may be disconnected from the capsule and be carefully pulled out of the patient, after which the capsule continues out of the patient by the natural route.

In the following, the invention will be described in further detail with reference to the drawing, wherein:

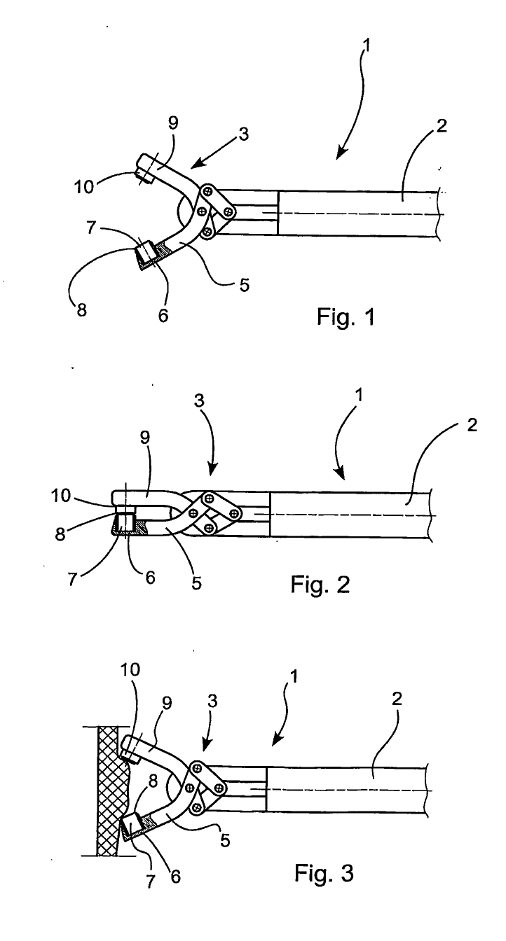

FIG. 1 shows biopsy forceps with exchange and replaceable receptacle, wherein the arm holding the receptacle is shown in partially sectional view;

FIG. 2 shows the forceps of FIG. 1 in closed position;

FIG. 3 shows the forceps just before a sample is taken;

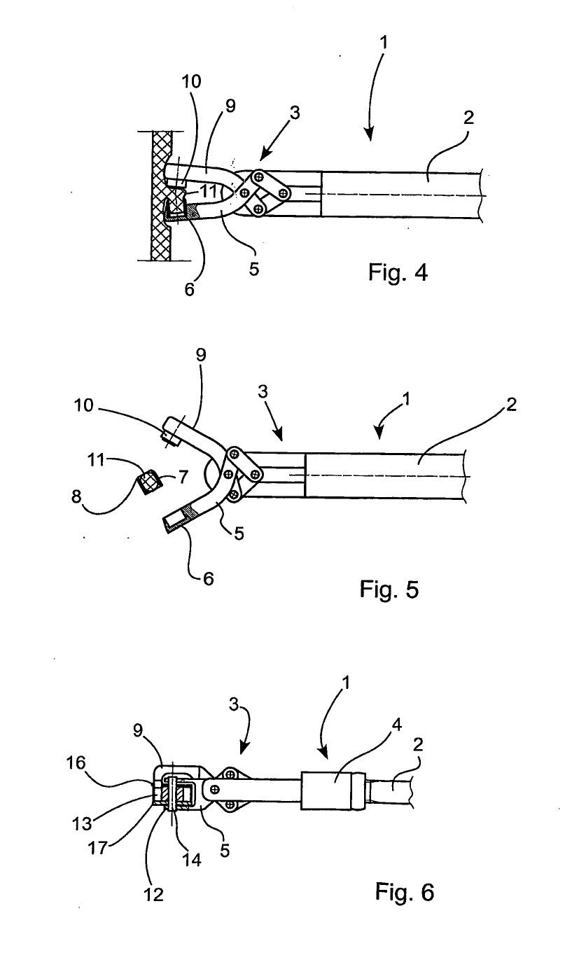

FIG. 4 shows the forceps during sampling;

FIG. 5 shows the forceps when the sample has been taken and the receptacle has been released from the forceps;

FIG. 6 shows an embodiment of the biopsy forceps with a turnable drum or cylinder with several chambers;

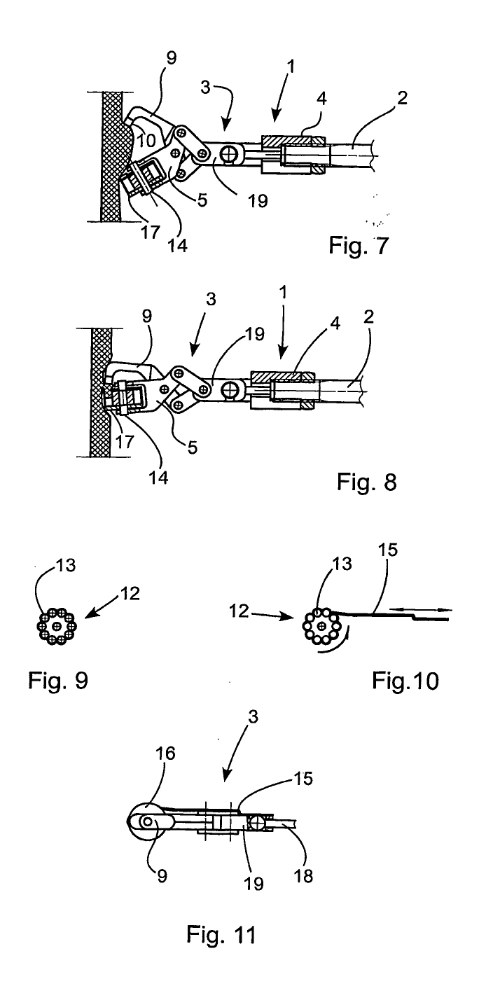

FIG. 7 shows the forceps of FIG. 6 just before a sample is taken;

FIG. 8 shows the forceps of FIG. 7 during sampling;

FIG. 9 shows a drum or cylinder with several chambers for the forceps;

FIG. 10 schematically shows the advancement mechanism for advancing an empty chamber; and

FIG. 11 shows the forceps shown in FIG. 7, seen from above, with the advancement mechanism.

Now convenient embodiments of the invention will be described. Biopsy forceps 1, used primarily for taking tissue samples and the like in the context of use of a kind of examination or treatment telescope (not shown) in the treatment of humans or animal's, comprise a kind of manoeuvring unit 2 in the form of a cable or the like, which cable 2 comprises at least an inner portion 18 and at least an outer portion that can be moved relative to each other to the effect that some kind of tool 3 arranged at the end of the cable 2 which is, via the telescope, conveyed into a patient can be operated by influencing one or more operating handles arranged at that end of the cable 2 that is arranged at the operating end of the telescope. A telescope for exercising such samplings usually comprises a number of passages in addition to a passage for the operating cable 2 or the sampling cable which, in combination with the tool 3, constitutes a biopsy cable or biopsy forceps 1. For instance, those passages are intended for sucking or flushing liquid and the like. At the end of the telescope, more specifically the end which is intended for being conveyed into the patient, a camera is arranged, whereby it is possible to see how the telescope is to be moved during insertion, and how the biopsy forceps 1 are to be moved to take the intended sample in the right place.

At the end of the cable 2 where the sample is to be taken, either a tool for sampling 3 is arranged, or coupling means 4 are provided for connecting and disconnecting a tool, which coupling means may be of ordinary type. The most important aspect in this context is that a tool 3 can be arranged at the end of the cable 2 in such a manner that it is easy to replace if, in some scenario or other, there is a need for securing another type of tool 3 to the cable 2 and for the tool 3 to be secured onto the cable 2 in a reliable manner without a risk of it falling off during use.

The biopsy forceps 1 are, in the part of the forceps that that performs cutting or shearing, provided with an exchange in the form of two mutually hinged arms 5, 9, where the first arm 5 comprises a holder device 6 for attaching a receptacle part 7 which receptacle part 7 is, at the end that faces away from the arm 5 in which the receptacle 7 is secured, provided with a cutting means 8. Another arm 9 which, during the sampling, cooperates with the first arm 5 with the receptacle 7, is provided with some kind of stop means or abutment 10 that combines with the cutting means 8 on the receptacle 7 to cut off or shear off the tissue sample 11.

The receptacle 7 may be configured as a cylinder that is arranged and secured in a corresponding opening 6 in the first arm 5. The opening 6 for attachment of the receptacle 7 may be closed at the bottom, but it may also be open where there is an edge or collar along the edge of the opening 6 at that end of the opening that faces away from the other arm 9. In those cases where the opening 6 is not closed at the bottom, the bottom may be replaced by some kind of sieve. This sieve may be a plate that is provided with a number of holes or in the shape of a grating. According to one embodiment of the opening, where the bottom is closed, the bottom may be provided with a number of holes.

According to another embodiment, that kind of sieve may be configured at the end of the cylindrical receptacle 7, which is the end that faces away from the end which is provided with cutting means 8.

The sieve serves the purpose of withholding the samples 11 that are taken with the biopsy forceps 1 to the effect that they do not fall out of the one end of the receptacle 7.

Interiorly, the receptacle may be provided with a surface that resembles sharkskin, that is with a number of relatively small one-way tips that protrude from the surface at an angle to the effect that samples, once they have entered the receptacle by movement along the sides of the receptacle, are directed towards the end of the receptacle 7 that faces away from the receptacle opening.

Moreover the stop means or abutment 10 of the biopsy forceps 1 may be arranged above the opening of the receptacle 7, once one has finished taking samples, and hence it will serve as lid.

This can be accomplished in that, when no more samples are to be taken, the forceps 1 can be locked in closed position during withdrawal.

The abutment 10 can be configured to be essentially plane, but it may be provided with an elevation at the middle which, when the forceps 1 are closed, extends into the receptacle at least past the cutting edge of the cutting means 8. According to another embodiment of the abutment 10, it may be configured with an indentation at the centre to the effect that, when the forceps 1 are closed, the receptacle 7, or at least the cutting edge of the cutting means 8, extends into to the abutment 10.

When the biopsy forceps 1 has been removed either from the telescope or along with the telescope, the receptacle 7 with the samples 11 can be removed from the forceps 1. This can be accomplished by means of tongs, optionally adapted for this particular function. The most important aspect of such tongs for removing the receptacle 7 is that the tongs have a secure grip around the receptacle 7 to avoid that the samples 11 are wasted or polluted, eg if the receptacle 7 is let go.

The receptacle 7 may also be provided with engagement means that engage with cooperating means in the arm 5 of the biopsy forceps 1. For instance, a bayonet socket, a thread or a tightly fitting connection optionally with a magnet for additional attachment may be used. The attachment of the receptacle 7 may also be accomplished by the cylindrical wall of the receptacle being slotted in a longitudinal direction, whereby the receptacle 7 is, when arranged, squeezed together and hence secured in the arm 5 on the forceps 1.

According to another embodiment of the biopsy forceps 1, the receptacle may be formed from some kind of drum or cylinder 12 that constitutes a magazine to the effect that the magazine 12 can be turned following a sample 11 has been taken such that a new chamber 13 is caused to be in position for another sample 11 to be taken and arranged in an empty chamber 13. This procedure can be repeated until there are no more empty chambers 13 in the magazine 12.

Here the sampling takes place in the same manner in that the arm 9 with some kind of abutment 10 presses the sample 11 against a cutting means, whereby the sample is cut off and arranged in a chamber 13.

The drum 12 may rotate about a shaft 14 that secures the drum 12 to the first arm 5. A latch, leaf spring 15 or the like is coupled to the inner cable 18 and advances or turns the drum 12 one storage space or one chamber 13 each time the forceps 1 are opened completely. The drum 12 may furthermore be provided with a spring-biased lock that ensures that the drum 12 does not turn unintentionally.

The advantage of a flat configuration is that the latch 15 is thereby controlled by the tool/biopsy forceps 1 to the effect that the latch 15 does not capsize when the advancement of the magazine 12 is activated.

In other cases, a simple rigid material may be used, for instance the end of a cable 2 can be used optionally provided with some kind of guide or slide bushing in proximity of the point of attack, where the cable 2 influences the magazine 12.

To ensure that the samples 11 do not fall out of the charged chambers 13 there may be arranged a disc or plate 16 above the magazine 12 that covers the chambers 13 in the magazine 12 with the exception of precisely the chamber 13 that has been positioned to receive the next sample 11. When the last sample 11 has been taken, the forceps 1 are kept closed during withdrawal of the endoscope. The upper cover plate or disc 16 may, by means of a sharp edge at its opening, form a cutting means for the sampling. The upper cover plate 16 is secured on the shaft 14 relative to the first arm 5 such that the cover plate 16 does not turn relative to the arm.

A lower cover plate 17 is arranged between drum 12 and the first arm 5. This cover plate 17 may either rotate along with the drum 12 or be fixed relative to the first arm 5.

According to a particular embodiment the magazine 12 may be configured such that, at least between two chambers 13, there is a distance that essentially corresponds to the diameter of a chamber 13. Thereby the magazine 12 may, following ended sampling, be parked in a position where precisely that area is arranged just opposite the opening in the upper plate or disc 16 to the effect that all of the samples 11 that are taken remain in their respective chambers 13 when the biopsy forceps 1 are taken out of the telescope or removed from the patient along with the telescope.

The advancement mechanism 15 for the magazine 12 may be provided with a locking mechanism ensuring that the magazine 12 can be advanced in only one direction.

The advancement mechanism can be driven by the inner cable 18 or by some other mechanism.

When the desired number of samples have been taken, the samples 11 may be advanced from the magazine 12 into a storage receptacle with formalin or other tissue-preserving preparation, or the entire magazine 12 may be dismounted and arranged in formalin or other tissue-preserving preparation in which the samples 11 may be stored until further examination takes place.

It applies to all embodiments that they may be connected to a cable by means of a coupling 4, where the forceps are secured to the outer part of the manoeuvring cable 2 by means of a snap coupling, bayonet socket, thread or the like, and wherein the inner cable 18 that slides within the outer cable, is secured to an intermediate piece 19 to the arms of the forceps in the same manner as the way in which a handbrake cable is secured to the brake handle of a bike or the like.

Thereby various tools may be used in connection with a cable, and in that a bore is provided at the end of the telescope that is, during use, located inside the patient, which bore is larger than the passage in which the cable usually travels, it is possible to mount the tool on the cable after the cable has been taken through the passage in the telescope.

However, the invention is not limited to that embodiment, some tools allowing, due to their size, that they can be conveyed through the passage in the telescope along with the cable after the telescope has been arranged inside the patient.

By providing a bore or the like measure to increase the diameter of the passages within the telescope at that end of the telescope which is intended for being introduced into the patient, a kind of “garage” is provided in which the tool 3 can be stored during introduction and withdrawal of the telescope into and from the patient in those cases where the coupling 4 on the cable is used for securing a tool 3 which is too large for travelling through the passage in the telescope.

The bore in the telescope is used to advantage in that the tool 3 is mounted on the cable 2 after the cable 2 has been taken through the cable passage. Then the cable 2 is pulled so far back that the tool 3 is completely or partially enclosed by the bore.

Obviously, the invention can be combined crosswise of the various embodiments of the invention described above.

In the context of the various embodiments of the tool 3, it is an option, at that end of the tool 3 that faces towards the coupling means of the cable, to provide connection for suction and/or flushing passages either via passages in the telescope or via connection at the manoeuvring end of the telescope or the biopsy cable.

The individual tools that can thus be arranged at the end of a biopsy cable may have different dimensions adapted in accordance with the task for which the tool is used.

1. Biopsy forceps for taking tissue samples and the like in connection with the use of some kind of examination or treatment telescope (1) in the treatment of humans or animals, characterised in that the part of the forceps (1) that performs cutting or shearing is provided with an exchange in the form of two mutually hinged arms (5, 9), wherein the first arm (5) comprises a holder device (6, 14) for securing a receptacle part (7, 12), which receptacle part (7, 12) is, at the end that faces away from the arm (5) in which the receptacle (7) is secured, provided with a cutting means (8); and wherein a second arm (9) which, during the sampling, cooperates with the first arm (5) with the receptacle (7) is provided with some kind of stop means or abutment (10) that cooperates with the cutting means (8) on the receptacle (7, 12) to cut off or shear off the tissue sample (11).

2. Biopsy forceps according to claim 1, characterised in that the biopsy forceps (1) are provided with an exchangeable receptacle (7, 12) for storing one or more tissue samples (11).

3. Biopsy forceps according to claim 1, characterised in that the biopsy forceps (1) is provided with a container (12) for taking several tissue samples (11), which receptacle (12) comprises a number of chambers (13) for storing one or more samples (11).

4. Biopsy forceps according to claim 3, characterised in that the receptacle (12) is configured with a number of chambers (13) arranged along the circumference of the container and around the axis of rotation of the receptacle (12).

5. Biopsy forceps according to claims 1-4, characterised in that the inner surface of the receptacle (7) or the chamber (13) is provided with one-way protrusions for retaining in the container (7) or the chamber (13) a collected sample (11).

6. Biopsy forceps according to claims 1-5, characterised in that, at the end of a cable (2), coupling means (4) are provided for exchangeably securing a tool (3) for use in connection with the cable (2).

7. Biopsy forceps according to claims 1-6, characterised in that the receptacle (7) or the chamber (13) is provided with perforations at the end that faces away from the cutting means (8).

8. Biopsy forceps according to claims 1-7, characterised in that the receptacle (7) or the chamber (13) is provided with a kind of grating at the end that faces away from the cutting means (8).

9. Biopsy forceps according to claims 3-8, characterised in that the receptacle (12) is configured as a turnable drum or cylinder comprising a plurality of chambers (13) for collecting samples (11).

10. Biopsy forceps according to claim 9, characterised in that the biopsy forceps (1) are provided with an advancement mechanism (15) for advancing a chamber to sampling position by turning of the drum or the cylinder (12).
